(19) United States
(12) Patent Application Publication
(10) Pub. No.: US 2026/0325484 A1
(43) Pub. Date: Oct. 22, 2026

(54) LONG-ACTING PAIN RELIEF FORMULATION

(71) Applicants: Centre National de la Recherche Scientifique, Paris (FR); Institut National de la Santé et de la Recherche Médicale, Paris (FR); Université d'Angers, Angers (FR); Nantes Université, Nantes (FR); Université Paris-Saclay, Gif-Sur-Yvette (FR)

(72) Inventors: Ruxandra GREF, Verrieres Le Buisson (FR); Laurent MARSOLLIER, Avrille (FR); Estelle MARION, Avrille (FR)

(21) Appl. No.: 19/484,783

(22) PCT Filed: May 15, 2024
(86) PCT No.: PCT/EP2024/063404

§ 371 Date: Nov. 14, 2025

(30) Foreign Application Priority Data
May 16, 2023 (FR) FR2304875

(51) Int. Cl. A61K 31/365; A61K 9/19; A61K 9/51; A61K 47/40; A61P 25/04

(52) U.S. Cl. CPC A61K 31/365; A61K 9/19; A61K 9/5161; A61K 47/40; A61P 25/04

(57) ABSTRACT

The present invention relates to a composition comprising a β-cyclodextrin polymer (a); and at least one compound (b) selected from mycolactone and its derivatives.

The present invention relates to a composition comprising a β-cyclodextrin polymer and at least one compound selected from mycolactone and its derivatives, and its uses, in particular for the treatment of pain.

Worldwide, more than 100 million people suffer from chronic pain associated with numerous conditions (osteoarthritis, cancer, lower back pain, sciatica, shingles, neuropathic pain, burns). In particular, neuropathic pain, resulting from direct damage to sensory neurons, is resistant to most treatments. Pain is one of the most difficult symptoms for patients to treat and endure. Currently, there are many treatments available to relieve pain, particularly those based on the use of morphine derivatives. These treatments have many limitations, such as short duration of action, a narrow therapeutic window, significant adverse side effects (nausea, constipation, balance disorders, drowsiness, addiction) and an increased risk of causing dependence in patients. There is therefore a real need to find alternatives that provide effective, long-lasting pain relief.

The use of mycolactone and its derivatives as analgesics appears to be a promising alternative. Indeed, application WO 2015/189342 describes the use of these molecules for the treatment of pain and their mechanism of action as agonists of the angiotensin 2 type 2 receptor (AT₂R) receptor agonists, type I cyclooxygenases and potassium channels of the TRAAK family. This involves hyperpolarisation of neurons, preventing the transmission of nerve signals responsible for pain to the brain. This use therefore has the advantage of not inducing dependence in patients.

Nevertheless, the use of this powerful analgesic is largely limited by the chemical nature of mycolactone derivatives, which makes them difficult to administer. These polyketide derivatives are extremely sensitive to light and therefore degrade rapidly. Furthermore, their chemical nature makes them completely insoluble in aqueous media and requires the use of an organic solvent for administration. Finally, mycolactone and its derivatives have low diffusion in the body once administered.

The use of nanoparticles or polymers to facilitate the administration of particularly poorly soluble pharmaceutical agents is currently an important area of medical research. Gouveia et al. Nature Scientific Reports 7, 5390 (2017) describe the use of β-cyclodextrin nanoparticles to encapsulate ethionamide (ETH), which is used in particular for the treatment of tuberculosis. The incorporation of the active ingredient also preserves its activity, improves the release of the agent into the lungs and therefore makes tuberculosis treatment more effective. However, this formulation has only been tested for administration into the lungs.

There is therefore a need to provide new, more effective treatments for pain.

There is also a need to find a suitable composition that allows for the gradual and prolonged release of mycolactone and its derivatives, while protecting them from degradation and avoiding their potentially toxic effects.

The present invention meets these needs in particular.

Indeed, as demonstrated in the examples, the inventors have surprisingly discovered that the combination of a β-cyclodextrin polymer with a molecule chosen from mycolactone or its derivatives makes it possible to solubilise mycolactone or its derivatives in an aqueous medium without the use of an organic solvent. Furthermore, it advantageously protects mycolactone from degradation under UV irradiation.

Therefore, a first object of the present invention is a composition comprising:
(a) a β-cyclodextrin polymer; and
(b) at least one compound selected from mycolactone and its derivatives.

A second object of the present invention is a method for preparing said composition.

Another object of the present invention is a pharmaceutical composition comprising at least the composition according to the invention and at least one pharmaceutically acceptable excipient.

Another object of the present invention is a composition for use in the treatment of pain.

The composition according to the invention comprises a compound (a) which is a β-cyclodextrin polymer.

The term “β-cyclodextrin polymer”, refers to all compounds that can be obtained by polymerisation of a compound selected from the β-cyclodextrin family, or by grafting a compound selected from the β-cyclodextrin family onto a (co)polymer.

The term “cyclodextrin” has a common meaning in the prior art and refers to a family of cyclic oligosaccharides, consisting of macrocyclic compounds comprising several glucopyranose subunits linked together by a 1,4-glycosidic bond.

Cyclodextrins are mainly produced by enzymatic degradation of amylose and its derivatives.

The cyclodextrins mainly used are divided into three families: α-, β- and γ-cyclodextrins. These families are distinguished by the number of units that make up the cyclic structure of cyclodextrins. “α-Cyclodextrin” is composed of 6 glucopyranose groups in its cyclic structure, “β-cyclodextrin” is composed of 7 glucopyranose groups in its cyclic structure, and “γ-cyclodextrin” is composed of 8 glucopyranose groups in its cyclic structure.

The chemical structure of β-cyclodextrin is as shown in formula (A).

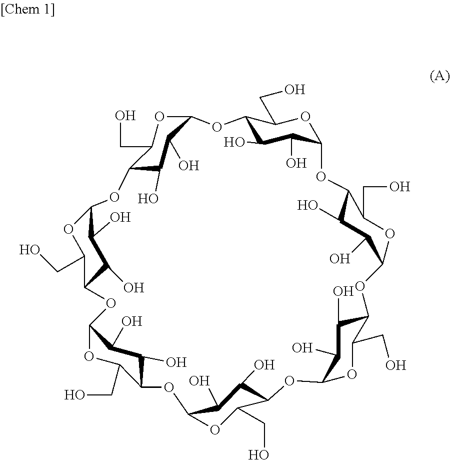 [SMILES: OCC1O[C@@H]2O[C@@H]3C(CO)O[C@H](O[C@@H]4C(CO)O[C@H](O[C@@H]5C(CO)O[C@H](O[C@@H]6C(CO)O[C@H](O[C@@H]7C(CO)O[C@H](O[C@@H]8C(CO)O[C@H](O[C@H]1[C@H](O)C2O)C(O)[C@H]8O)C(O)[C@H]7O)C(O)[C@H]6O)C(O)[C@H]5O)C(O)[C@H]4O)C(O)[C@H]3O]

β-cyclodextrin molecules have a truncated cone structure, delimiting a cavity in their centre. This cavity has a non-polar, hydrophobic carbon environment (carbon skeleton and oxygen in ether bonds), capable of accommodating poorly water-soluble molecules, while the outside of the truncated cone has numerous hydroxyl groups, leading to good solubility of β-cyclodextrin in aqueous media. This amphiphilic nature also allows β-cyclodextrin to include hydrophobic molecules (or parts thereof) in its cavity to form inclusion complexes in aqueous media.

The formation of these inclusion complexes makes it possible to encapsulate various molecules, in particular mycolactone and possibly other molecules of therapeutic interest, in β-cyclodextrin molecules. Advantageously, a cyclodextrin polymer comprises more than twenty β-cyclodextrin cages, not all of which are occupied by mycolactone. This makes it possible to co-incorporate a variety of active molecules of interest in the family of antibiotics, analgesics, anticancer drugs, etc.

The term “inclusion complex” refers to a system composed of a host molecule (in this case, β-cyclodextrin) capable of accommodating a chemical species (in this case, mycolactone and its derivatives).

The term “encapsulate” indicates that the chemical species is included within the host molecule.

According to one embodiment, the β-cyclodextrin polymer (a) is prepared by a polymerisation reaction between modified or unmodified β-cyclodextrin and at least one other chemical compound selected from the group consisting of epichlorohydrin, tartaric acid, citric acid, acylated poly(ethylene glycol), adipoyl chloride, succinyl chloride, glutaraldehyde, diphenyl carbonate, 1,4-butanediol diglycidyl ether, toluene diisocyanate, naphthalene diisocyanate, succinic anhydride, 1,2,4,5 benzene tetracarboxylic anhydride, monochlorotriazine, trimethoxysilane derivatives, diethynyl benzene, tetrafluoro terephtalonitrile. Another type of cyclodextrin polymer is obtained from cyclodextrin-based organic-inorganic porous particles (metal-organic frameworks, MOFs), which can then be cross-linked using the agents mentioned above in this paragraph.

Alternatively, water-soluble copolymers can be grafted with cyclodextrins. These copolymers are among the group consisting of: alginates, poly(ethyleneimine), poly(N-hydroxyethyl acrylamide), chitosan, hyaluronic acid, phenylalanine, polyanhydrides, polyaspartamide, and cellulose.

According to another embodiment, polymer (a) is the product of the reaction between modified or unmodified β-cyclodextrin and epichlorohydrin.

According to another embodiment, polymer (a) is the product of the reaction between modified β-cyclodextrin and epichlorohydrin.

“Modified β-cyclodextrin” refers to any β-cyclodextrin that has been modified by at least one chemical reaction without altering the intrinsic properties of β-cyclodextrin.

Chemical reaction refers in particular to addition reactions, substitution reactions, or even polymer grafting reactions.

In general, the modified β-cyclodextrins used according to the invention are, in particular, β-cyclodextrins whose hydroxy groups, preferably the secondary hydroxy groups of each glucose unit forming them, have been modified by the addition of one or more identical or different substituents.

A “modified β-cyclodextrin” used in the composition according to the invention is a β-cyclodextrin that carries one or more identical or different substituents, chosen from functionalised or non-functionalised alkyl radicals, hydroxyalkyl radicals, carboxyl radicals, carboxylates, nitro, amino, sulphonates, sulphates, phosphates, ethers, polyethers, ammonium and radicals comprising an ester function. An alkyl radical may be a linear or branched alkyl radical having 1 to 20 carbon atoms, preferably 1 to 5 carbon atoms, more particularly a methyl or ethyl radical. As a hydroxyalkyl radical, we can cite a hydroxyalkyl group having 1 to 20 carbon atoms, preferably 1 to 5 carbon atoms, more particularly a —CH₂CH₂OH or —CH₂OH. radical. As a radical containing an ester function, an ester group having 1 to 20 carbon atoms, preferably 1 to 5 carbon atoms, more particularly the groups —O(CO)CH₃, —O(CO)CH₂CH₃, may be mentioned

Examples include methylated, ethylated, propylated, succinylated, carboxylated, acetylated, 2-hydroxypropylated and polyoxyethylated cyclodextrins.

Preferably, polymer (a) is a cross-linked polymer, i.e. the polymer has a three-dimensional, non-linear network structure formed by the creation of bonds between the macromolecular chains of a polymer during the polymerisation reaction. This polymer can then be modified to add new functionalities such as negative or positive charges, or by grafting fluorescent molecules.

Preferably, polymer (a) is the product of the reaction between β-cyclodextrin and epichlorohydrin.

According to this embodiment, this polymer is known and described by Gref et al. Journal of Controlled Release: Official Journal of the Controlled Release Society 2006, 111 (3), 316-324 under the name pCD.

Polymers (a) can be prepared by adapting or applying the method described by Gref et al. Journal of Controlled Release: Official Journal of the Controlled Release Society 2006, 111 (3), 316-324, and Othman et al., J Colloid Interface Sci 2011, 354 (2), 517-27.

More specifically, a polymer (a) can be prepared by polymerisation reaction between β-cyclodextrin and epichlorohydrin in an alkaline medium in the presence of NaOH. For example, β-cyclodextrin is dissolved under vigorous stirring in a solution comprising 33% by mass of NaOH, then epichlorohydrin is added under stirring and the reaction is stopped by adding acetone before the gelation point.

According to this method of production, the chemical structure of polymer (a) is as represented by general formula (B):

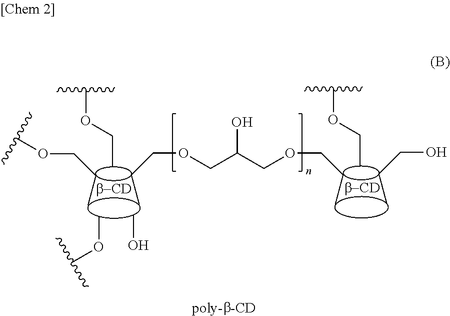

in which the truncated cones represent the β-cyclodextrin molecules and n represents a repeating unit corresponding to the epichlorohydrin molecules that have reacted and separate two β-cyclodextrin molecules.

The composition according to the invention comprises a compound (b) selected from mycolactone and its derivatives.

Mycolactone and its derivatives are 12-membered polyketide derivatives originally produced by various bacterial strains of the Mycobacterium ulcerans (Mu) family, which are involved in Buruli ulcers (BU).

Compounds (b) are readily available.

They can, for example, be obtained by extraction and purification from Mycobacterium ulcerans extract. This method is described in George et al; SCIENCE Volume 283, Issue 5403, 5 Feb. 1999.

It is possible to use different strains of Mycobacterium ulcerans that produce different isomers. The production of isomers depends on the area from which the bacterial strain originates.

The mycolactone used in these examples comes from strain 1615, which corresponds to the bibliographic reference cited in George et al; SCIENCE Volume 283, Issue 5403, 5 Feb. 1999. It is also possible to extract mycolactone from strains obtained directly from patients.

Alternatively, the compounds can be synthesised. A synthetic method for preparing mycolactone and its derivatives has been developed and described in application EP 2 594 561.

According to one embodiment, compound (b) is a compound of formula (I):

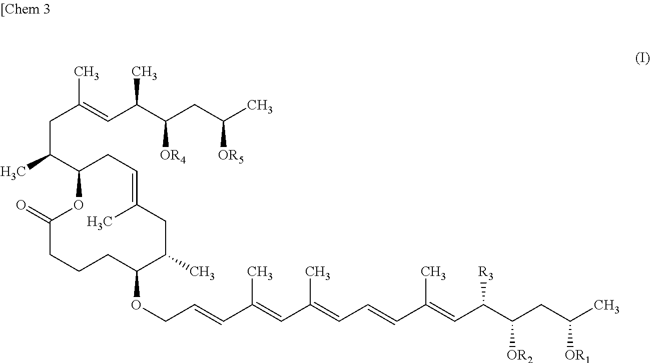

wherein:
   R₁, R₂, R₄ and R₅ are identical or different and are independently selected from the group consisting of H, R₆, C(O)R₇, C(S)R₇, C(O)NHR₇ and C(S)NHR₇,
   R₃ is selected from the group consisting of H, OH, OR₆, OC(O)R₇, OC(S)R₇, OC(O)NHR₇, OC(S)NHR₇ and OCH(OH)R₇,
   R₆ is a group selected from the group consisting of C₁-C₆ alkyl, C₆-C₁₂ aryl, C₆-C₁₂ heteroaryl and sugar derivatives, and
   R₇ is selected from the groups H, C₁-C₆ alkyl, C₆-C₁₂ heteroaryl, and C₆-C₁₂ aryl, where
   R₁ and R₂, R₂ and R₃, and/or R₄ and R₅ together form an acetal group,
as well as its pharmaceutically acceptable salts.

The term “C₁-C₆ alkyl” group refers to a linear or branched aliphatic hydrocarbon group comprising, unless otherwise specified, a total of 1 to 6 carbon atoms, and which may comprise one or more unsaturations. Examples include methyl, ethyl, n-propyl, butyl, isobutyl, tert-butyl, pentyl and hexyl groups.

The term “C₆-C₁₂ aryl” group refers to monocyclic, bicyclic or tricyclic aromatic hydrocarbon compounds comprising, unless otherwise specified, a total of 6 to 12 carbon atoms. Examples include phenyl and naphthyl groups.

The term “C₆-C₁₂ heteroaryl” group refers to one of the monocyclic, bicyclic or tricyclic aromatic compounds comprising 6 to 12 carbon atoms in total, at least one atom of which is a heteroatom chosen from the group consisting of nitrogen, phosphorus, oxygen and sulphur. Examples include pyrrole, furan, thiophene, imidazole, furazane, oxazole, oxadiazole, oxatriazole, isoxazole, thiazole, isothiazole, pyrazole, triazole and tetrazole groups. The term “sugar derivative” refers to compounds comprising at least one sugar unit selected from mono- and polysaccharides. Examples of monosaccharide units include glucose, galactose, fructose and pentose.

According to another embodiment, compound b) is a compound of formula (II):

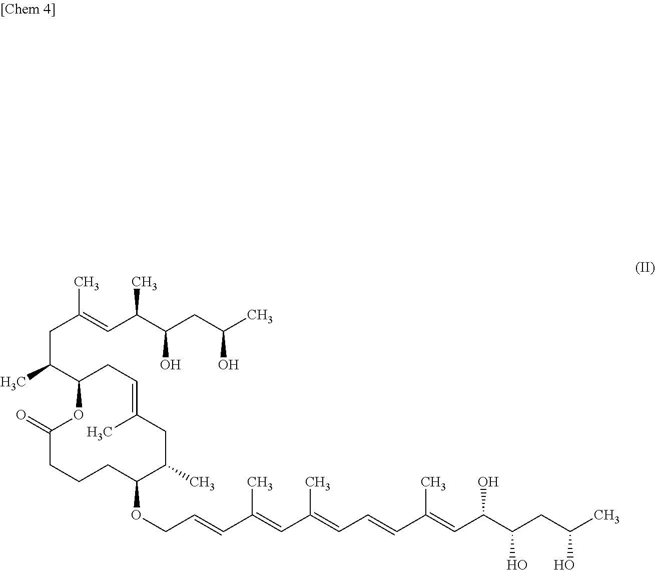 [SMILES: CC(=C\C=C\C(C)=C\[C@H](O)[C@@H](O)C[C@H](C)O)/C=C(C)/C=C/CO[C@H]1CCCC(=O)O[C@@H]([C@@H](C)C/C(C)=C/[C@@H](C)[C@H](O)C[C@@H](C)O)C/C=C(\C)C[C@@H]1C]

and its pharmaceutically acceptable salts.

This compound is a compound of formula (I) in which R₁, R₂, R₄ and R₅ are —H groups and R₃ is an —OH group; it corresponds to mycolactone.

According to one embodiment, the composition according to the invention comprises a compound (b) which is encapsulated in the β-cyclodextrin polymer (a). By encapsulated in the polymer (a) we mean the incorporation of mycolactone by solubilisation in an aqueous medium. In practice, this is achieved by adding an aqueous solution of β-cyclodextrin polymer (a) to a container containing pure mycolactone. Upon contact with (a), the mycolactone is solubilised, with or without mechanical agitation. Alternatively, to accelerate the process, sonication or an Ultraturrax may be used.

Without wishing to be bound by any particular theory, the molecules of mycolactone and/or its derivatives form inclusion complexes with the β-cyclodextrin molecules that make up polymer (a). The formation of these inclusion complexes also allows the compounds (b) to be encapsulated within the β-cyclodextrin molecules that make up the polymer.

Advantageously, the formation of these inclusion complexes allows the compounds (b) to be spontaneously solubilised without the use of an organic solvent.

Advantageously, the encapsulation of the compounds (b) in the β-cyclodextrin polymer (a) also significantly stabilises the compounds (b) against degradation, particularly under UV irradiation.

Advantageously, the very strong affinity of mycolactone and its derivatives for β-cyclodextrin allows prolonged release of compound (b) into the body after administration of the composition.

In other words, the composition according to the invention is a composition comprising a β-cyclodextrin polymer in which compound (b) is encapsulated.

Preferably, the composition according to the invention is in powder form or may be in aqueous solution, even more preferably the composition is in aqueous solution.

In particular, the powder can be obtained by freeze-drying said composition according to the invention in aqueous solution.

Advantageously, the encapsulation of compounds (b) by the β-cyclodextrin polymer surprisingly allows an aqueous composition with a high concentration of compound (b) to be obtained.

According to one embodiment, the composition is an aqueous solution in which the concentration of compound (b) is between 0.001 mg/mL and 10 mg/mL, preferably between 0.05 and 5 mg/mL.

Preferably, the composition according to the invention is an aqueous solution in which the concentration of compound (a) is between 25 mg/mL and 250 mg/mL, preferably between 50 mg/mL and 150 mg/mL.

Preferably, the composition according to the invention comprises a ratio of the amount of compound (b) in weight percent to the amount of polymer (a) in weight percent that can be from 0.1 to 10, preferably from 0.5 to 7, and more preferably from 1 to 5, the percentages being expressed relative to the total weight of the composition.

According to another embodiment, the β-cyclodextrin polymer (a) can typically encapsulate at least one other active ingredient having, for example, a complementary, potentiated or synergistic action with compound (b). Generally, the active ingredients are encapsulated within the cavities of the free β-cyclodextrin molecules forming polymer (a), i.e. not already being inclusion complexes with a compound (b).

According to one embodiment, the composition according to the invention may further comprise one or more active ingredients (c) selected from anti-cancer agents, analgesic agents, anaesthetic agents, active molecules with a healing effect, antibacterial agents, antibiotics, antiseptics, haemostatics, antifungals, antivirals, antithrombotics, anti-inflammatories, antipruritics, contrast agents, and hormones.

Typically, the active ingredient is an antiseptic, such as chlorhexidine or benzalkonium chloride, ethanol, hexamidine, betadine, chlorinated derivatives, triclocarban.

In another embodiment, the active ingredient (c) is an analgesic agent, such as lidocaine, codeine, tramadol.

According to another embodiment, the active ingredient (c) is an antibiotic agent such as vancomycin (VCM), amikacin, gentamicin, or amoxicillin.

According to another embodiment, the active ingredient (c) is an anticancer agent such as doxorubicin, cyclophosphamide, cisplatin, docetaxel, gemcitabine, or oxaliplatin.

Compound (b) and said active ingredients may be present in the form of a hydrate and/or a pharmaceutically acceptable salt. Indeed, these compounds may be present in the form of corresponding salts of pharmaceutically acceptable organic or inorganic acids or organic or inorganic bases.

The term “pharmaceutically acceptable salts” refers to relatively non-toxic inorganic and organic acid addition salts and base addition salts of the compounds of the present invention. These salts may be prepared in situ during the final isolation and purification of the compounds. In particular, acid addition salts may be prepared by separately reacting the purified compound in its purified form with an organic or inorganic acid and isolating the salt thus formed. Examples of acid addition salts include hydrobromate, hydrochloride, sulphate, bisulphate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptanate, lactobionate, sulfamates, malonates, salicylates, propionates, methylene bis-b-hydroxynaphtoates, gentisic acid, isethionates, di-p-toluoyltartrates, methanesulfonates, ethanesulfonates, benzenesulfonates, p-toluenesulphonates, cyclohexyl sulphonates and quinates, laurylsulphonate, and analogues. (See, for example, S. M. Berge et al. “Pharmaceutical Salts” J. Pharm. Sci, 66: p. 1-19 (1977)). Acid addition salts may also be prepared by separately reacting the purified compound in its acid form with an organic or inorganic base and isolating the salt thus formed. Acid addition salts include amino and metal salts. Suitable metal salts include sodium, potassium, calcium, barium, zinc, magnesium and aluminium salts. Sodium and potassium salts are preferred. Suitable inorganic base addition salts are prepared from metal bases which include sodium hydride, sodium hydroxide, potassium hydroxide, calcium hydroxide, aluminium hydroxide, lithium hydroxide, magnesium hydroxide, zinc hydroxide. Suitable basic amino addition salts are prepared from amines that have sufficient alkalinity to form a stable salt, and preferably include amines that are often used in medicinal chemistry due to their low toxicity and acceptability for medical use: ammoniac, ethylenediamine, N-methylglucamine, lysine, arginine, ornithine, choline, N,N′-dibenzylethylenediamine, chloroprocaine, diethanolamine, procaine, N-benzyl-phenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g. lysine and arginine, and dicyclohexylamine, and analogues.

According to another embodiment, the β-cyclodextrin polymer (a) can advantageously be combined with other compounds such as lipid compounds (cholesterol, injectable oils such as Migliol), glycoproteins (such as lamins), nanoparticles (NPs), thereby advantageously conferring complementary properties to the composition according to the invention, such as antibacterial or antifungal properties, targeting properties, or the inclusion of other active ingredients in the composition.

This encapsulation is generally feasible if the size of said nanoparticles allows their inclusion within the cavities of free β-cyclodextrin molecules.

Preferably, the size of the nanoparticles (d) is between 50 nm and 400 nm, more preferably between 50 nm and 250 nm. The lipid compounds can form microdroplets ranging in size from 50 nm to 2-5 microns within the formulation.

According to another object, the present invention also relates to a pharmaceutical composition comprising the composition according to the invention and at least one pharmaceutically acceptable excipient.

The pharmaceutical composition of the invention may be presented in various forms intended for topical, parenteral or intrathecal administration. Preferably, said composition is administered to a patient who needs it.

Topical administration means the administration of a product to a surface of the body such as the skin or mucous membranes.

Parenteral administration refers to the administration of a product by means of an injection. Parenteral administration includes intramuscular (IM), intravenous (IV), subcutaneous (SC), intradermal, intra-arterial or intra-articular injections.

Intrathecal administration refers to an injection into the subarachnoid space to reach the cerebrospinal fluid, which in turn will diffuse the injected product.

Injectable pharmaceutical forms, particularly for parenteral or intrathecal use, are generally injectable solutions that can be obtained, for example, by the following process: the composition according to the invention is dissolved, suspended or emulsified in an aqueous medium (e.g. distilled water, physiological saline or Ringer's solution), with a dispersant (e.g. Tween® 80, HCO® 60 (Nikko Chemicals), polyethylene glycol, carboxymethylcellulose or sodium alginate), a preservative (e.g. methyl p-hydroxybenzoate, propyl p-hydroxybenzoate, benzyl alcohol, chlorobutanol or phenol), an isotonic agent (e.g. sodium chloride, glycerol, sorbitol or glucose) and optionally other additives, such as, if desired, a solubilising agent (e.g. sodium salicylate or sodium acetate) or a stabiliser (e.g. human serum albumin).

Pharmaceutical forms for topical use can be obtained from a solid, semi-solid or liquid composition containing the composition of the invention. For example, to obtain a solid form, the composition according to the invention may be mixed with excipients (e.g. lactose, trehalose, sucrose, mannitol, starch, microcrystalline cellulose or sucrose) and a thickener (e.g. natural gums, cellulose derivatives or acrylic polymers) in order to transform them into a powder. Liquid pharmaceutical compositions are prepared in much the same way as injectable forms, as described above. Semi-solid pharmaceutical forms are preferably in the form of aqueous or oily gels or in the form of ointments. These compositions may optionally contain a pH regulator (e.g. carbonic acid, phosphoric acid, citric acid, hydrochloric acid or sodium hydroxide), an antioxidant compound (vitamin C, tocopherol, polyphenols) and/or a preservative (e.g. p-hydroxybenzoic acid ester, chlorobutanol or benzalkonium chloride).

According to one embodiment, the pharmaceutical composition according to the invention comprises a pharmaceutically acceptable excipient selected from dextran and its derivatives, preferably hydrophobised dextran.

Advantageously, the use of hydrophobised dextran in the pharmaceutical composition according to the invention allows the formation of gels or nanoparticles. In this case, it is possible to incorporate nanoparticles as described in Example 13 or oil droplets.

Preferably, the size of the nanoparticles (d) is between 50 nm and 400 nm, more preferably between 50 nm and 250 nm. The lipid compounds can form microdroplets ranging in size from 50 nm to 2-5 microns within the formulation.

According to one embodiment, the composition according to the invention may further comprise nanoparticles (d) selected from inorganic nanoparticles, organic-inorganic hybrid nanoparticles, or polymeric nanoparticles.

The term “nanoparticle” (NP) generally refers to a spherical particle with an average diameter (in number) between 1 nm and 1 μm. In the context of the invention, said NPs typically have an average diameter of less than 400 nm, in particular less than 250 nm, with a polydispersity index of less than 0.25.

The average diameter (in number) and polydispersity can be measured by dynamic light scattering (DLS) or transmission electron microscopy (TEM).

Examples of inorganic nanoparticles include magnetic nanoparticles, silver nanoparticles with antibacterial properties, gold nanoparticles and copper nanoparticles.

Organic-inorganic hybrid nanoparticles include MIL-100(Fe) iron trimetate particles, other iron carboxylates, and UIO-66.

Examples of polymeric nanoparticles include PLGA (poly(lactic-co-glycolic acid)), poly(ethylene glycol) (PEG)-PLGA (PEG-PLGA) copolymer, and polycaprolactone (PCL) particles.

Advantageously, the nanoparticles (d) may themselves include an active ingredient.

According to another object, the present invention also relates to a method for preparing the composition according to the invention, comprising a step of mixing compounds (a) and (b) in aqueous solution.

According to another embodiment, the process according to the invention may optionally comprise a step of adding an active ingredient (c) and/or nanoparticles (d) to the aqueous solution.

Preferably, the process according to the invention may also comprise a freeze-drying step to obtain said composition in powder form.

Another object of the present invention concerns the use of the composition according to the invention for the treatment of pain.

The term “pain” also refers to “painful disorders” and denotes more or less intense suffering caused by an injury, burn, lesion or any other cause, which manifests itself as a disruption of well-being, health balance, or loss or reduction of physical integrity.

Pain is generally classified as acute or chronic. “Acute” pain refers to a sudden, short-lived experience associated with a specific cause, such as a specific injury resulting, for example, from surgery, dental treatment or a sprain.

“Chronic pain” refers to long-term pain causing significant psychological and emotional problems. This type of pain is associated with many conditions, such as osteoarthritis, cancer and diabetes.

The composition according to the invention is suitable for the treatment of a wide range of painful disorders, including when these appear as symptoms of pathologies, in particular acute pain, chronic pain, neuropathic pain, inflammatory pain, iatrogenic pain, including cancer pain, infectious pain, including herpetic pain, visceral pain, central pain, dysfunctional pain, including fibromyalgia, nociceptive pain, including post-surgical pain, and mixed types of pain involving the viscera, gastrointestinal tract, cranial structures, musculoskeletal system, spine, urogenital system, the cardiovascular system, and the CNS, including cancer pain, back pain, and orofacial pain.

In general, pain disorders are considered symptoms of a pathology that must be treated independently of the pathology itself for the patient's well-being.

The term “symptom” refers to an abnormal manifestation caused by a pathology of which the patient complains.

The term “pain management” used here refers to the relief, inhibition of progression or elimination of pain disorders as described here, without necessarily treating the condition that may be responsible for them. Thus, pain management can typically be symptomatic and may possibly be aetiological.

In general, pain or “pain disorders” in patients are usually treated by administering short-acting analgesics or painkillers.

The term “analgesic” refers to drugs used to suppress sensitivity to pain, such as opioid derivatives.

“Analgesics” refers to drugs used to reduce pain.

Preferably, the composition according to the invention is used as an analgesic to treat pain or painful disorders.

According to another embodiment, the composition according to the invention is used as an analgesic to treat pain and painful disorders.

Advantageously, the composition according to the invention can be used in a complementary manner to treat painful disorders caused by a pathology.

### FIGURES

FIG. 1. A: Improvement in the encapsulation of mycolactone (ML) with an increase in the amount of pCD. B: successive extractions of ML from ML-pCD.

FIG. 2. Protective role of pCD against degradation of mycolactone exposed to UV light.

FIG. 3. A) No additional cytotoxic effect caused by pCD. B-D) pCD does not alter the immunomodulatory effects (production of IL-6, TNF-α, and IL-1β) of mycolactone. Black=free ML; grey=incorporated ML (pCD-ML)

FIG. 4. Analgesic effect of mycolactone encapsulated in pCD in mice (#: difference between ML and pCD-ML).

FIG. 5. Biodegradable cyclodextrins reduce the analgesic effect of mycolactone compared to pCD.

FIG. 6. Analgesic effect of mycolactone encapsulated in pCD in a plantar incisional model in mice (surgical pain model) (#: difference between ML and pCD-ML).

FIG. 7. Analgesic effect of mycolactone encapsulated in pCD in a plantar incisional model in mice (surgical pain model) (#: difference between ML and pCD-ML).

### EXAMPLES

### Example 1—Synthesis and Characterisation of a Cyclodextrin Polymer (pCD)

In a 500 mL flask, 50 g of β-CD is added to 80 mL of 33% NaOH and left to stir overnight. The next day, the flask is heated to 30 °C and 35 mL of epichlorohydrin (EP) is added while stirring (Scheme 1). The reaction mixture is kept under stirring at 30 °C for 1 to 1.5 hours to ensure it is thoroughly homogenised. The reaction is stopped when the viscosity increases, just before the setting point, by adding 80 mL of acetone.

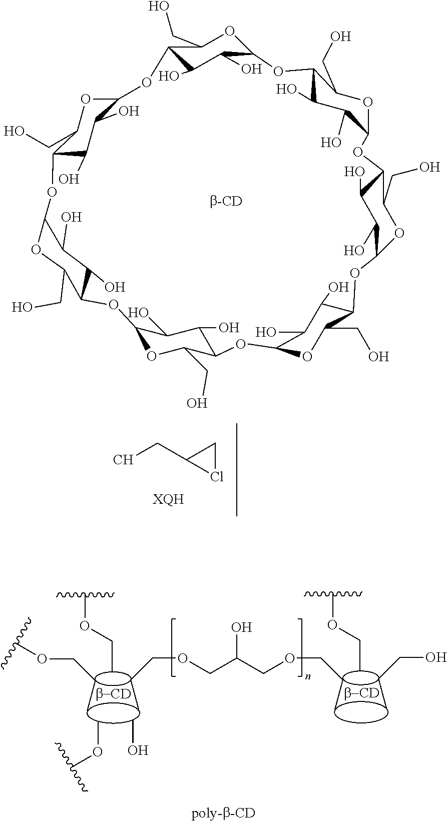

Diagram 1. Reaction diagram for the synthesis of pCD from β-CD cross-linked with epichlorohydrin.

The bath temperature is raised to 50 °C and left to stir overnight. The next day, neutralise with a 6 M HCl solution to obtain a pH of 7. Remove the insoluble residue, then purify the remaining polymer by dialysis using a Spectra/por membrane (cut-off threshold 100,000 g/mole) and freeze-dry. The pCD polymer thus obtained is characterised by NMR and SEC to determine its β-CD content, which is around 70% (wt.).

Using the same methodology, aα-CD polymer and aγ-CD polymer are synthesised. pCD samples can be fractionated by successive dialysis (using, for example, SpectraPor membranes with cut-off thresholds of 20, 50, 100, 300 and 1000 KDa) in order to obtain samples with low polydispersity.

### Example 2—Synthesis and Characterisation of a Cyclodextrin Polymer Based on Malic Acid

In a 25 mL flask, 0.2 mmol NaH2PO4·2H2O (catalyst), 0.09 mmol β-CD, and 0.45 mmol malic acid are mixed in 2 mL water. The mixture is then concentrated t e by evaporation at 140 °C for 10 min and then heated to 140 °C for 25 min under reduced pressure (vacuum pump). The polymer thus formed is taken up with 10 mL of MilliQ water and dispersed by ultrasound. The insoluble fraction is removed by filtration. The soluble fraction is then purified by dialysis (Spectra/por membrane, cut-off threshold 20,000 g/mole) and then lyophilised. The polymer is then characterised by (^{1) H}NMR and SEC to determine its β-CD content, which is around 70% (wt.).

This polymer is called pCD.

A biodegradable β-CD polymer is synthesised in a similar manner, replacing malic acid with citric acid.

### Example 3—Synthesis of Dextran Modified with Alkyl Chains

To synthesise dextran grafted with hydrophobic lauryl chains (DM), 4 g of dextran (40,000 g/mol) was solubilised in 100 mL of dimethylformamide containing 1 g of lithium chloride. Next, 0.62 mL of lauryl chloride and 0.031 mL of pyridine were added to the dextran solution. The reaction was carried out at 80 °C for 3 hours. The MD obtained was isolated by precipitation in isopropyl alcohol. It was then solubilised in distilled water, purified by dialysis for 48 hours and then lyophilised. The grafting rate of the alkyl chains determined by (^{1)H}NMR is around 6%.

### Example 4—Production of Mycolactone

Mycolactone is purified from a culture of M. ulcerans strain 1615 according to the protocol described by Georges 1999 (1). Briefly, total mycobacterial lipids are extracted using the Folch method. Then, after precipitation of the phospholipids in cold acetone, the supernatant containing mycolactone is deposited on a silica plate for thin-layer chromatography. After migration, the silica onto which the mycolactone has adsorbed (rf of 0.23) is scraped off and the mycolactone is desorbed from the silica by filtration (in chloroform/methanol). Finally, the mycolactone is quantified by high-performance liquid chromatography (2).
1. George K M, Chatterjee D, Gunawardana G, Welty D, Hayman J, Lee R, Small P L. 1999. Mycolactone: a polyketide toxin from Mycobacterium ulcerans required for virulence. Science 283:854-857.
2. Marion E, Prado S, Cano C, Babonneau J, Ghamrawi S, Marsollier L. 2012. Photodegradation of the Mycobacterium ulcerans toxin, mycolactones: considerations for handling and storage. PLoS One 7:e33600.

### Example 5—Incorporation of Mycolactone into pCD

0.5 mg of ML (mycolactone) solubilised in ethanol is placed in a 1.5 mL amber glass vial. The ethanol is evaporated using a vacuum concentrator (SpeedVac). 1 mL of pCD solution at a concentration of 100 mg/mL is then added. The preparation is stirred for 48 hours and then stored at 4 °C. This allows time for complete incorporation of the ML into the pCD. This solution is called pCD-ML.

The experiment is repeated, replacing the pCD with a β-CD polymer and a β-CD polymer. The ML does not solubilise, even when the incubation time is extended to 72 hours.

### Example 6—Improvement in the Encapsulation of Mycolactone by Increasing the Amount of pCD

Starting with a 100 mg/mL pCD solution, serial dilutions are made in MilliQ water to obtain the following pCD concentrations: 1, 10, 50 and 100 mg/mL.

In a 1.5 mL amber glass vial, 0.5 mg of ML solubilised in ethanol is introduced. The ethanol is evaporated using a vacuum concentrator (SpeedVac). Then, 1 mL of pCD solution at concentrations of 1, 10, 50 and 100 mg/mL is added. The preparation is shaken for 48 hours.

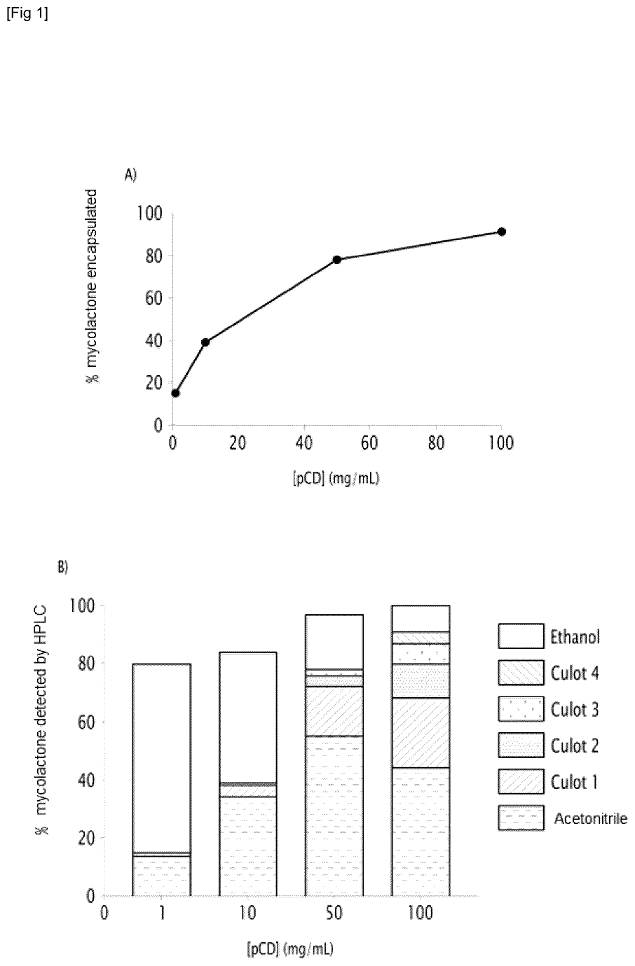

Aliquots are recovered and measured by HPLC as in Example 5 to determine the amount of ML incorporated. It is found that at least 100 mg/mL of pCD is required to incorporate 0.5 mg of ML (FIG. 1 A).

After incubation with stirring, 30 μL of each of the pCD-ML preparations are taken and diluted to 1/10^{th} in acetonitrile. After centrifugation (3000 g, 5 min), the supernatants are measured by HPLC. All pellets are recovered in MilliQ water and diluted 1/10^{th} in acetonitrile before being centrifuged and measured again by HPLC. These steps are repeated three times in order to extract all the encapsulated ML content at the different pCD concentrations tested.

The rest of the preparation is transferred to ethanol and then measured by HPLC after being diluted to 1/10th in acetonitrile and centrifuged (3000 g, 5 min).

In conclusion, the higher the ML content in the pCD, the more difficult it becomes to extract the ML from the pCD. Thus, four successive extractions are necessary to extract all of the ML from the pCD (100 mg/mL of pCD and 0.5 mg of incorporated ML). This demonstrates the strong affinity of ML for pCD. It is highly likely that at high concentrations, the highly hydrophobic ML can self-associate within the pCD (FIG. 1 B).

### Example 7—Stability of pCD-ML During Storage

Surprisingly, ML incorporated into pCD no longer adsorbs onto glass or plastic containers, making it possible to handle. This was demonstrated by measuring the concentration of ML by HPLC in a pCD-ML solution prepared as in Example 5. After 3 months, there was less than 4% variation in concentration.

### Example 8—Protective Role of pCD Against the Degradation of Mycolactone Exposed to UV light

50 μL of pCD-ML solution prepared according to Example 5 (corresponding to 0.5 mg/mL of ML) are placed in transparent glass tubes. The tubes are then exposed to UV irradiation (room temperature, wavelength 312 nm) for 15 minutes, 1 hour, 2 hours, 6 hours, and 24 hours. One tube is kept in the dark and serves as a control. All tubes are sealed tightly to prevent evaporation.

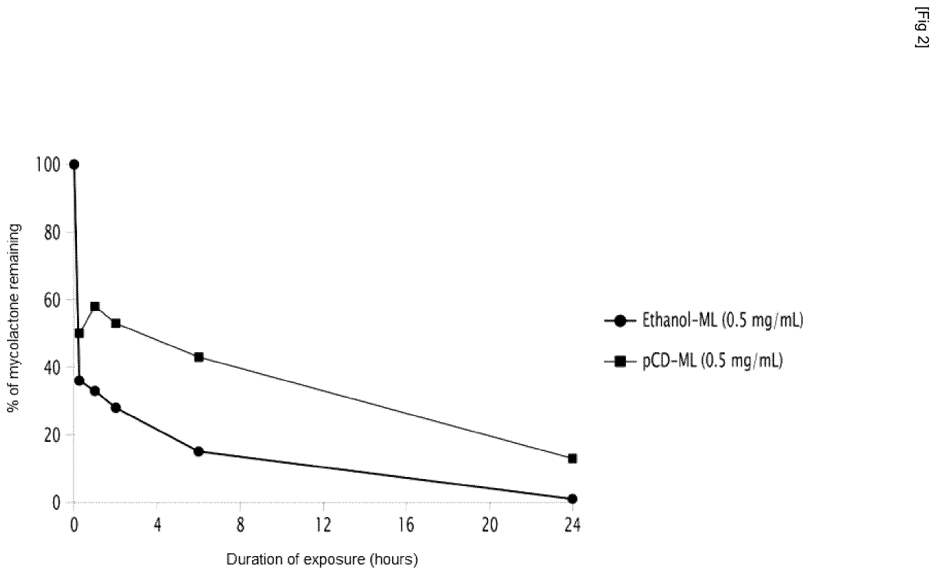

After these different periods of exposure to UV light, the ML is measured by HPLC. A series of ML extractions is carried out as described in example 6. To do this, 450 μL of acetonitrile are added to each tube containing 50 μL of pCD-ML. After 6 hours of exposure, 80% of the ML contained in ethanol had been degraded, whereas over the same period only 50% of the ML associated with pCD had been degraded (FIG. 2).

### Example 9—In Vitro Evaluation of the Cytotoxic and Immunomodulatory Effects of pCD on Murine Macrophages

Two preparations were made: an ethanolic solution of ML and an aqueous solution of pCD-ML at 0.5 mg/mL. Cascade dilutions were performed to obtain solutions with concentrations of 100-10-1 μg/mL and then 60-20-2 ng/mL in ML. 100 μL of each dilution are distributed per well containing 100 μL of cells (100,000 cells/well), giving a final concentration of 30-10-1 ng/mL of ML. The cells were incubated for 24 hours (at 37 °C, 5% CO₂), then stimulated with 20 μL of a lipopolysaccharide (LPS) solution at 500 ng/mL, giving a final concentration of 50 ng/mL LPS in each well. After 24 hours of incubation, the supernatants were collected.

To determine the cytotoxic effect of ML, the Lonza™ ToxiLight™ test (Fisher Scientific) is used. To do this, 5 μL of each supernatant is mixed with 25 μL of reagent.

To determine the immunomodulatory effect of ML, ELISA kits (IL-6, TNF-α, IL-1b) were used, after diluting the supernatants 1/10^{th} for the IL-6 and TNF-α kits, and ½ for the IL-1b kit.

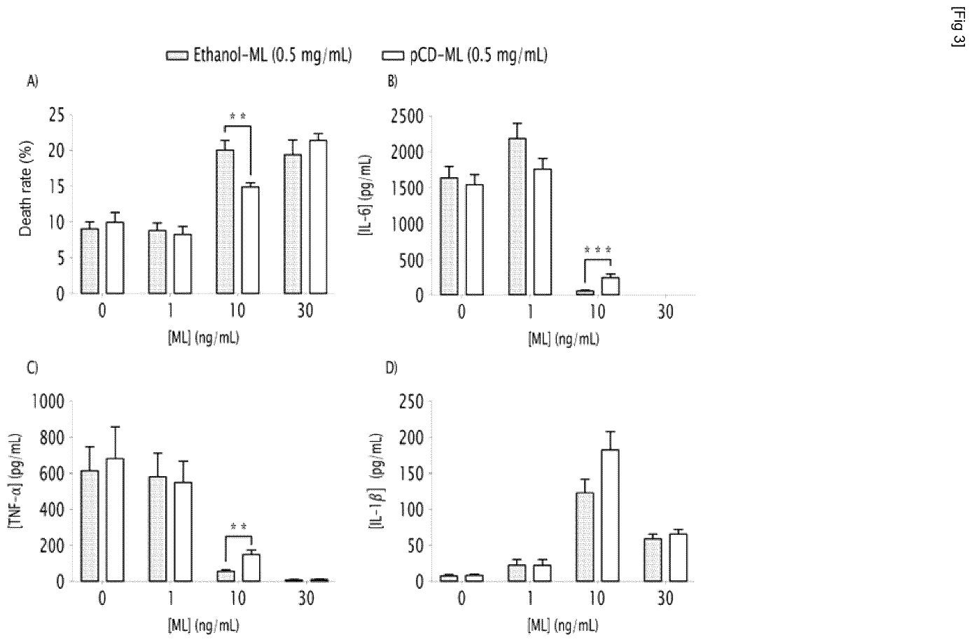

It appears that the incorporation of ML into pCD does not significantly alter its immunomodulatory effect (FIG. 3. A-D).

### Example 10—Analgesic Effect of Mycolactone Encapsulated in pCD in Mice

In three different tubes, solutions of pCD in water (100 mg/mL), ML in ethanol (3 mg/mL) and pCD-ML (3 mg/mL) are prepared as described above. The pCD was then diluted with physiological saline to a concentration of 8.33 mg/mL. The ML was diluted in corn oil (1) to a concentration of 250 μg/mL. Finally, the pCD-ML preparation was diluted with physiological saline to an ML concentration of 250 μg/mL.
1. Babonneau J, Breard D, Reynaert M L, Marion E, Guilet D, Saint Andre J P, Croue A, Brodin P, Richomme P, Marsollier L. 2019. Mycolactone as Analgesic: Subcutaneous Bioavailability Parameters. Front Pharmacol 10:378.

The experiments were conducted on female Balb/c mice aged 7 to 10 weeks. 15 μL of the preparation was injected subcutaneously into the paw pad. The latency time for the mice to withdraw their paw following a thermal stimulus was then measured using the Hargreaves plantar testing device. The value expressed corresponds to the ratio of the test group to the control group (pCD alone).

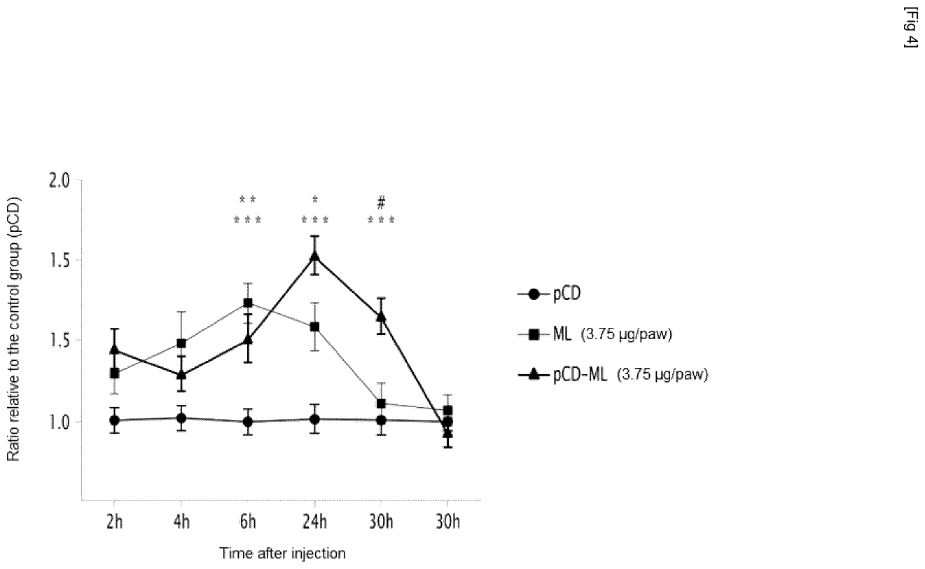

As shown in FIG. 4, the analgesic effect induced by pCD-ML is greater than that of ML alone, and this effect lasts longer. For statistical analysis, a two-factor ANOVA followed by a Dunnett multiple comparison test was used: *p<0.05, p**<0.01, ****p<0.0001)).

### Example 11—Biodegradable Cyclodextrins Reduce the Analgesic Effect of Mycolactone

Solutions with a concentration of 100 mg/mL were prepared from pCD and biodegradable cyclodextrins (called pCD-citrate). ML was encapsulated in each of these solutions at a concentration of 3 mg/mL. These preparations were then diluted in physiological saline to a concentration of 250 μg/mL. At the same time, control solutions without ML were prepared in the same way.

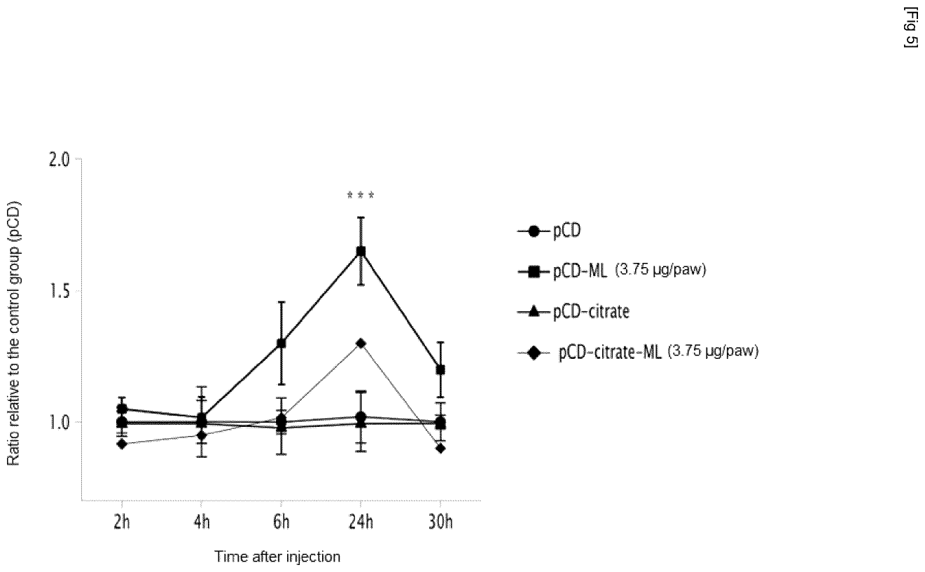

These experiments are performed on Balb/c mice aged 7 to 10 weeks. 15 μL of the preparation is injected subcutaneously into the paw pad. The latency time for the mice to withdraw their paw following a thermal stimulus is then measured using the plantar test apparatus by the Hargreaves method. The value expressed corresponds to the ratio of the test group to the control group (pCD alone). As shown in FIG. 5, the analgesic effect induced by pCD-ML is greater than that of ML alone, and this effect lasts longer. For the statistical analyses, a two-factor ANOVA was used, followed by a Dunnett multiple comparison test *p<0.05, p**<0.01, ****p<0.0001). Under our conditions, only the incorporated ML has a significant analgesic effect. This result shows the specificity of ML-pCD in inducing an analgesic effect.

### Example 12—Analgesic Effect of Mycolactone Encapsulated in pCD in a Plantar Incisional Model in Mice (Surgical Pain Model)

In two different tubes, solutions of pCD in water (100 mg/mL) and pCD-ML (3 mg/mL) are prepared as described above. These solutions were then diluted with physiological saline to obtain final preparations containing 3.75 μg of ML, 1.875 μg of ML and 0.937 μg of ML per 15 μL of solution.

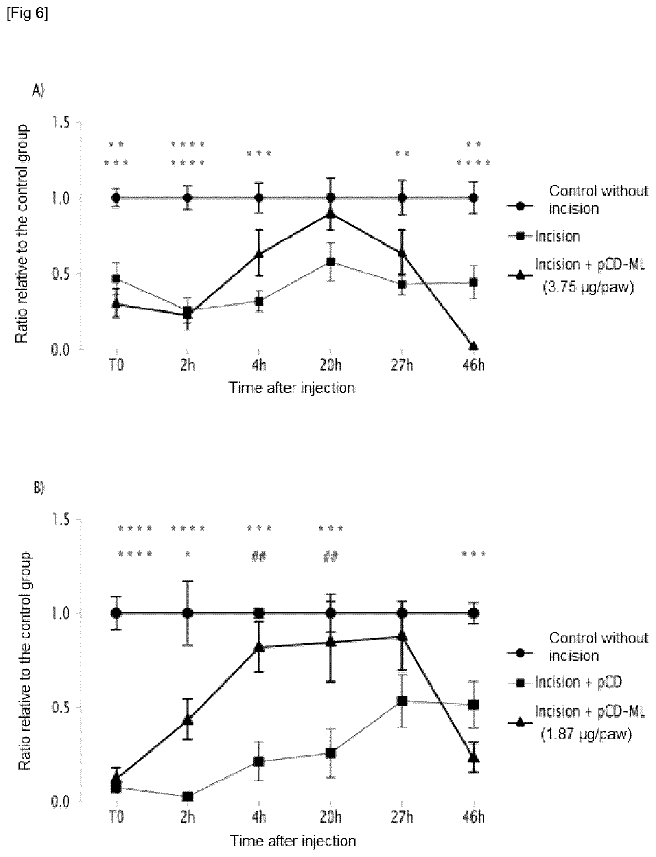

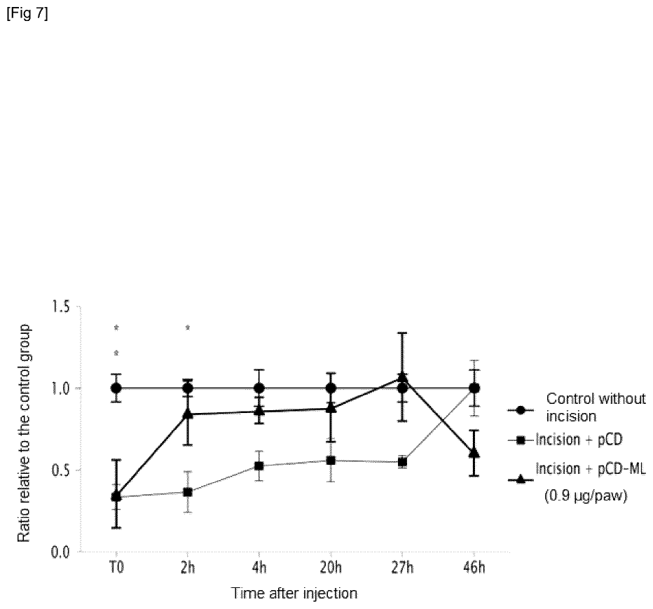

The value expressed corresponds to the ratio of the test group to the control group (pCD alone). As shown in FIGS. 6 and 7, the analgesic effect induced by pCD-ML is greater than that of ML alone and this effect lasts longer. For statistical analysis, a two-factor ANOVA followed by a Dunnett multiple comparison test was used: *p<0.05, p**<0.01, **** p<0.0001)). Under our conditions, only ML-pCD has a significant analgesic effect. This result shows the specificity of ML-pCD in inducing an analgesic effect.

These experiments were performed on C57Bl/6 mice aged 7 to 10 weeks. Incisions were made on the flexor digitorum brevis muscle. 15 μL of the preparation was injected subcutaneously as close as possible to the incision. The latency time for the mice to withdraw their paw following a thermal stimulus was then measured using the Hargreaves plantar test apparatus. The results obtained show that up to a dose of 0.9 μg ML in pCD (FIG. 7), a significant analgesic effect was observed more than 30 hours after application. The effect is more lasting for doses <3.75 μg of ML (FIG. 6. A). This result is surprising but can be explained by the fact that at low doses there is no longer any adverse effect (immunomodulatory effect) of ML that could reduce its analgesic effect.

### Example 13—Co-Incorporation of Magnetic Particles

A DM solution and a pCD solution are prepared, each at 75 mg/mL. Mixing equal volumes of these solutions instantly produces a soft gel system that settles at the bottom of the container. This gel can be recovered and passed through a syringe (20G needle).

The same operation is performed, except that 0.5 mg of iron oxide nanoparticles (Aldrich, <5 microns) ref 310069 are incorporated into the DM (1 mL) and left to incubate for 4 hours. After mixing with 1 mL of pCD, a dark-coloured gel system is formed. It can be moved using a magnet as it contains magnetic particles. Almost all of the magnetic particle s are incorporated into this gel. Furthermore, the system retains its cohesion even when significantly diluted (addition of 3 L of water).

The same experiment is carried out by incorporating ML into pCD as in example 5 and then mixing with the DM solution containing the magnetic particles. The same type of gel is formed.

### Example 14—Development of Nanoparticles

Identical volumes of DM (10 mg/mL) and pCD (10 mg/mL) are mixed with or without ML. Nanoparticles of approximately 120 nm are instantly formed, and a cloudy solution is observed.

### Example 15: Lyophilisation

Aqueous solutions of pCD-ML are prepared as described previously in Example 5, in amber vials. The final concentrations of pCD are 10 and 100 mg/mL, and those of ML are 0.3 and 0.5 mL, respectively. These solutions are frozen directly at −80 °C and then lyophilised (typically using an Alpha 1-2 LD Plus lyophiliser, −65 °C, 0.018 mbar, 24 h).

At the end of lyophilisation, a white solid is obtained. The pCD-ML solutions are easily reconstituted by adding water to this lyophilisate.

The freeze-dried form protects ML from light degradation better than the solubilised form. Advantageously, the lyophilisate can be stored for more than 8 months away from light without detectable degradation of the incorporated ML. The integrity of the ML incorporated into the pCD is determined by HPLC (Agilent, with a C18 Kinetex® 5 μm×250 mm×4.6 mm column; 100 Å). Detection is performed at 30 °C, at a wavelength of 363 nm, with a flow rate of 1 mL/min and an injection volume of 20 μL. The gradient is: 0 min=90/10 water/acetonitrile; 3 min=50/50 water/acetonitrile; 13 min=0/100 water/acetonitrile; 23 min=0/100 water/acetonitrile: 33 min=90/10 water/acetonitrile.

1. A composition comprising:
(a) a β-cyclodextrin polymer; and
(b) at least one compound selected from mycolactone and its derivatives.

2. The composition according to claim 1, wherein compound (b) has the following formula (I): 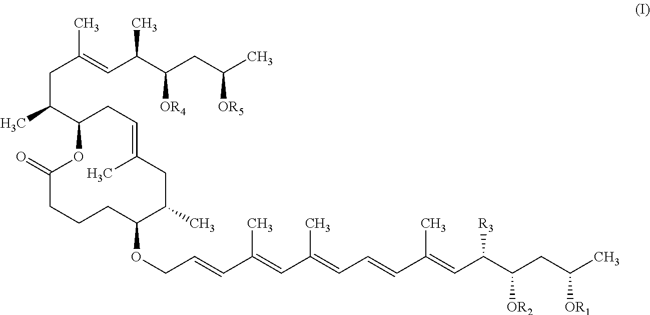
wherein:
R₁, R₂, R₄ and R₅ which may be identical or different, are independently selected from the group consisting of H, R₆, C(O)R₇, C(S)R₇, C(O)NHR₇ and C(S)NHR₇,
R₃ is selected from the group consisting of H, OH, OR₆, OC(O)R₇, OC(S)R₇, OC(O)NHR₇, OC(S)NHR₇ and OCH(OH)R₇,
R₆ is a group selected from the group consisting of C₁-C₆ alkyl, C₆-C₁₂ aryl, and sugar derivatives, and
R₇ is selected from the groups H, C₁-C₆ alkyl and C₆-C₁₂ aryl, or
R₁ et R₂, R₂ and R₃, and/or R₄ and R₅ together form an acetal group,
or a pharmaceutically acceptable salt thereof.

3. The composition according to claim 1, wherein compound (b) is mycolactone corresponding to formula (II): 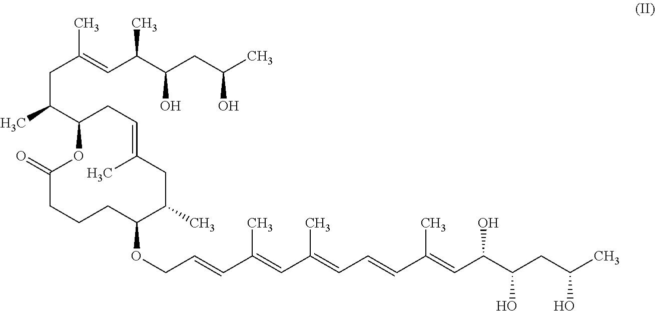 [SMILES: CC(=C\C=C\C(C)=C\[C@H](O)[C@@H](O)C[C@H](C)O)/C=C(C)/C=C/CO[C@H]1CCCC(=O)O[C@@H]([C@@H](C)C/C(C)=C/[C@@H](C)[C@H](O)C[C@@H](C)O)C/C=C(\C)C[C@@H]1C]
or a pharmaceutically acceptable salt thereof.

4. The composition according to claim 1, wherein compound (b) is encapsulated in the β-cyclodextrin polymer (a).

5. The composition according to claim 1, as present in powder form or in aqueous solution.

6. The composition according to claim 1, wherein the concentration of compound (b) is between 0.001 mg/mL and 10 mg/mL.

7. The composition according to claim 1, wherein polymer (a) is the product of the polymerisation reaction between modified or unmodified s-cyclodextrin and at least one other chemical compound selected from the group consisting of epichlorohydrin, tartaric acid, citric acid, acylated poly(ethylene glycol), adipoyl chloride, succinyl chloride, glutaraldehyde, diphenyl carbonate, 1,4-butanediol diglycidyl ether, toluene diisocyanate, naphthalene diisocyanate, succinic anhydride, 1,2,4,5-benzene tetracarboxylic anhydride, monochlorotriazine, trimethoxysilane derivatives, diethynyl benzene and tetrafluoro terephtalonitrile.

8. The composition according to claim 1, wherein polymer (a) is a polymer of β-cyclodextrin, modified or unmodified, cross-linked with epichlorohydrin.

9. The composition according to claim 1, further comprising an active ingredient (c) selected from anticancer agents, analgesic agents, anaesthetic agents, active molecules with a healing effect, antibacterial agents, antibiotics, antiseptics, haemostatics, antifungals, antivirals, antithrombotics, anti-inflammatories, antipruritics, contrast agents, and hormones.

10. The composition according to claim 9, further comprising comprising a compound (d) selected from inorganic nanoparticles, organic-inorganic hybrid nanoparticles or polymeric nanoparticles.

11. A pharmaceutical composition comprising the composition according to claim 1, and at least one pharmaceutically acceptable excipient.

12. The pharmaceutical composition according to claim 11, wherein the pharmaceutically acceptable excipient is selected from dextran and its derivatives.

13. The pharmaceutical composition according to claim 11, wherein the pharmaceutically acceptable excipient is hydrophobised dextran, leading to the formation of gels or nanoparticles.

14. A method for preparing the composition according to claim 1, comprising a step of mixing compounds (a) and (b) in aqueous solution.

15. A method for treatment of pain comprising administering the composition according to claim 1 to a patient in need thereof.

16. The composition according to claim 1, wherein the concentration of compound (b) is between 0.05 and 5 mg/mL.
